(19) United States
(12) Patent Application Publication
(10) Pub. No.: US 2018/0160967 A1
(43) Pub. Date: Jun. 14, 2018

(54) METHOD AND SYSTEM FOR DEALING WITH BREATHING ANOMALIES

(71) Applicant: Sensory Innovation Solutions, Inc., Marietta, GA (US)

(72) Inventors: Richard diMonda, Marietta, GA (US); Stephen F. Abbott, Redlands, CA (US)

(21) Appl. No.: 15/835,316

(22) Filed: Dec. 7, 2017

Related U.S. Application Data
(60) Provisional application No. 62/432,609, filed on Dec. 11, 2016.

(51) Int. Cl. A61B 5/00; A61H 31/00; A61B 5/08; A43B 3/30; A41D 19/01; A41B 13/00

(52) U.S. Cl. CPC A61B 5/4818; A61H 31/00; A61B 5/7282; A61B 5/0816; A43B 3/30; A61B 5/14542; A41B 13/00; A61H 2205/065; A61H 2205/125; A61H 2230/405; A61H 2230/425; A41D 19/01

(57) ABSTRACT

A system for dealing with breathing anomalies can monitor a subject for indications of breathing anomalies while the subject is sleeping. For example, the system can monitor blood oxygen saturation utilizing a pulse oximeter placed on a finger or toe, breathing rate utilizing a camera, or another appropriate physiological parameter. When a breathing anomaly is detected, the system can stimulate a volar surface of the subject, such as a planar surface of the subject's foot or a palmar surface of the subject's hand. The stimulation of the volar surface can manipulate the subject's breathing, for example producing the Babinski response as a sleep apnea therapy. Stimulating the volar surface can comprise producing or emulating a stroking motion across the volar surface, for example. In some examples, an array of actuators can be disposed adjacent the volar surface, and the actuators can actuate sequentially to stimulate the volar surface.

### CROSS REFERENCE TO RELATED APPLICATIONS

This application claims priority to U.S. Provisional Patent Application No. 62/432,609 filed Dec. 11, 2016 in the name of Richard diMonda and Stephen Abbott and entitled “Method and System for Tactile and Proprioceptive Stimulation for Sleep Apnea,” the entire contents of which are hereby incorporated herein by reference.

### TECHNICAL FIELD

Embodiments of the technology relate to controlling breathing and more specifically to producing tactile or proprioceptive stimulation to terminate, traverse, suppress, or avoid breathing anomalies, for example associated with sleep apnea.

### BACKGROUND

Many people suffer from breathing anomalies that occur during sleep or breathing-related sleep disorders. Representative examples include central sleep apnea (CSA) or central sleep apnea syndrome (CSAS), obstructive sleep apnea (OSA), chronic or habitual snoring, upper airway resistance syndrome (UARS), and obesity hypoventilation syndrome (OHS). It has been reported that almost half of low-birth-weight infants are sent home from the hospital with an apnea monitor. In addition to direct health consequences, such conditions can cause substantial anxiety for the afflicted and their caregivers and loved ones. For example, a parent of a sleeping infant may worry about the infant's breathing.

There is a deficiency in the art for technology to deal with breathing anomalies that may occur during sleep or rest. Need exists for an improved capability to predict or detect breathing anomalies and to correct breathing anomalies. Need further exists for an improved capability to terminate, traverse, or suppress a breathing anomaly early in the onset of the anomaly. Need further exists for an improved capability to preempt an anticipated breathing anomaly based on detection of a precursor to the anomaly. Need further exists for a capability to avoid occurrences of breathing anomalies. A technology addressing one or more such needs, or a related deficiency in the art, could improve sleep, breathing, and/or anxiety associated with breathing anomalies and breathing-related sleep disorders.

### SUMMARY

Tactile or proprioceptive stimulation can deal with breathing anomalies, for example to help preempt, terminate, traverse, overcome, or suppress a breathing anomaly that may be in progress or by providing a prophylactic that prevents occurrences of breathing anomalies.

In one aspect of the disclosure, a system can monitor a person for indications of breathing anomalies while the person is sleeping and take corrective action when a breathing anomaly is detected or anticipated. An example breathing anomaly can comprise a period of diminished breathing that may be associated with sleep apnea. To detect breathing anomalies, the system can monitor blood oxygen saturation utilizing a pulse oximeter placed on a finger or toe, breathing rate utilizing a camera, or another appropriate physiological parameter utilizing a suitable sensor, for example. When a breathing anomaly is detected or anticipated, the system can stimulate a volar surface of the person, such as a planar surface of his or her foot or a palmar surface of his or her hand. The stimulation can manipulate the subject's breathing, for example producing the Babinski response to preempt, prevent, terminate, suppress, manage through, or avoid a breathing anomaly. The subject's breathing can thus respond to the stimulation to correct the breathing anomaly.

In one aspect of the disclosure, stimulation of the volar surface of the person can comprise producing or emulating a stroking motion across at least a portion of the volar surface. An electromechanical system can produce or emulate a stroking motion, for example. In some examples, the electromechanical system can comprise an array of actuators disposed adjacent the volar surface, with the actuators actuating sequentially to stimulate the volar surface and produce a stroking sensation, which may trigger the Babinski response.

In one aspect of the disclosure, stimulation of the volar surface of the person can comprise sequentially stimulating at least two areas of the volar surface. The two areas can be stimulated with any or a combination of force or movement directed into or substantially normal to the volar surface, force or movement directed substantially along or transverse to the volar surface, friction, rubbing, pressure, vibration, sound, heat, electricity, electrical fields, magnetic fields, electromagnetic waves, light, or other appropriate energy form or motion, to mention some representative examples without limitation. The two areas may be contiguous, may adjoin one another, or may be separated by a gap in various examples.

The foregoing discussion of dealing with breathing anomalies is for illustrative purposes only. Various aspects of the present disclosure may be more clearly understood and appreciated from a review of the following text and by reference to the associated drawings and the claims that follow. Other aspects, systems, methods, features, advantages, and objects of the present disclosure will become apparent to those with skill in the art upon examination of the following drawings and text. It is intended that all such aspects, systems, methods, features, advantages, and objects are to be included within this description and covered by this paper and by the appended claims.

### BRIEF DESCRIPTION OF THE FIGURES

FIG. 1 is an illustration that includes a functional block diagram of a system for dealing with breathing anomalies, where the system is illustrated in a representative pediatric application, in accordance with some example embodiments of the disclosure.

FIG. 2 is an illustration of a breathing monitor that comprises a camera in accordance with some example embodiments of the disclosure.

FIG. 3 is an illustration of a wearable that is configured to be worn on a foot and that comprises a system for dealing with breathing anomalies in accordance with some example embodiments of the disclosure.

FIG. 4 is a functional block diagram of a system for dealing with breathing anomalies in accordance with some example embodiments of the disclosure.

FIG. 5 is a flowchart for a process for dealing with breathing anomalies in accordance with some example embodiments of the disclosure.

FIG. 6 is an illustration of a breathing stimulator that comprises an array of stimulation elements in accordance with some example embodiments of the disclosure.

FIGS. 7A, 7B, and 7C, collectively FIG. 7, are timing diagrams for individual stimulation elements of a breathing stimulator, where the breathing stimulator comprises an array of stimulation elements, in accordance with some example embodiments of the disclosure.

Many aspects of the disclosure can be better understood with reference to these figures. The elements and features shown in the figures are not necessarily to scale, emphasis being placed upon clearly illustrating the principles of example embodiments of the disclosure. Moreover, certain dimensions may be exaggerated to help visually convey such principles. In the figures, reference numerals designate like or corresponding, but not necessarily identical, elements throughout the several views.

### DETAILED DESCRIPTION OF EXAMPLE EMBODIMENTS

A system for dealing with breathing anomalies can comprise a computer-based machine. The computer-based machine can stimulate a volar surface of a subject to correct a breathing issue faced by the subject, for example alleviating diminished breathing associated with central sleep apnea. The computer-based machine can comprise electrically driven moving parts to implement the stimulation in some example embodiments. In some other example embodiments, the computer-based machine can implement the stimulation in a solid-state manner, without moving parts. In some example embodiments, the computer-based machine can monitor the subject's physiology to detect or predict onset of a breathing anomaly and take corrective action, so that the detection or prediction and the correction occur in a single sleep session, such as during one night. In some example embodiments, the computer-based machine can utilize findings from a prior sleep study to administer the stimulation in a matter that prevents onset of a breathing anomaly. For example, the computer-based machine can administer personalized stimulation on a pre-defined schedule, such as by administering a series of stimulations each night while the subject is sleeping.

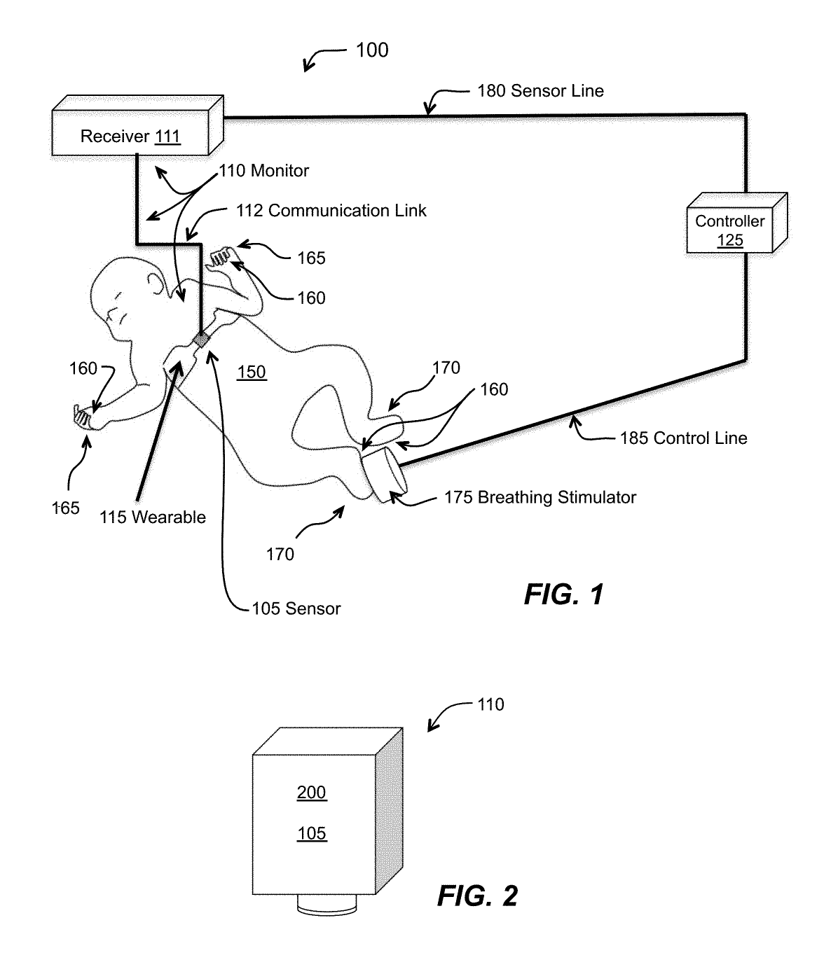

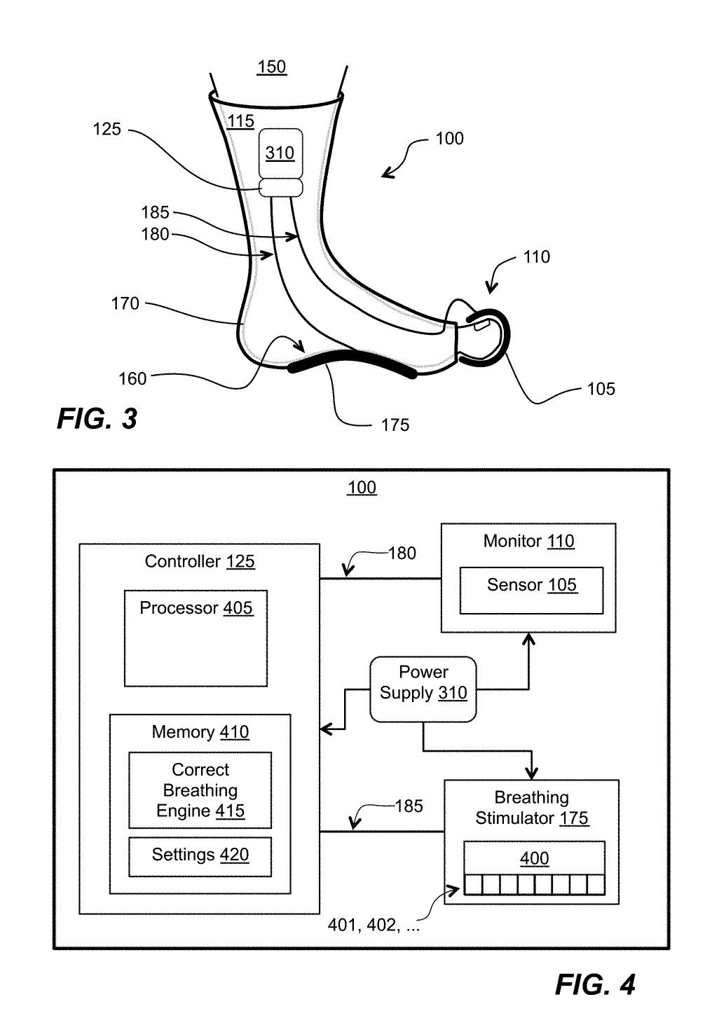

The technology will be discussed more fully hereinafter with reference to the Figures, which provide additional information regarding representative or illustrative embodiments of the disclosure. FIGS. 1 and 2 will be first discussed below, including in the example context of an embodiment that may be useful in a pediatric application. FIGS. 3, 4, 5, 6, and 7 will then be discussed, including in the example context of an embodiment comprising a wearable.

The present technology can be embodied in many different forms and should not be construed as limited to the embodiments set forth herein; rather, these embodiments are provided so that this disclosure will be thorough and complete, and will fully convey the scope of the technology to those having ordinary skill in the art. Furthermore, all “examples,” “embodiments,” and “exemplary embodiments” provided herein are intended to be non-limiting, and among others supported by representations of the disclosure.

This document includes sentences, paragraphs, and passages (some of which might be viewed as lists) disclosing alternative components, elements, features, functionalities, usages, operations, steps, etc. for various embodiments of the disclosure. Unless clearly stated otherwise, all such lists, sentences, paragraphs, passages, and other text are not exhaustive, are not limiting, are provided in the context of describing representative examples and variations, and are among others supported by various embodiments of the disclosure. Accordingly, those of ordinary skill in the art having benefit of this disclosure will appreciate that the disclosure is not constrained by any such lists, examples, or alternatives. Moreover, the inclusion of lists, examples, embodiments, and the like will help guide those of ordinary skill in practicing many more implementations and instances that embody the technology without undue experimentation, all of which are intended to be within the scope of the claims.

This disclosure includes figures and discussion in which features and elements of certain embodiments may be organized into functional blocks, subsystems, or modules. And, certain processes and methods may be organized into blocks or into steps. Such organization is intended to enhance readership and to facilitate teaching the reader about working principles of the technology and about making and using an abundance of embodiments of the disclosure. The organization is not intended to force any rigid divisions or partitions that would limit the disclosure. In practice, the flexibility of the technology and the depth of this disclosure supports dispersing or grouping functionalities, elements, and features in many different ways. The inclusion of an element or function in one block, module, or subsystem verses another may be substantially arbitrary in many instances, with the divisions being soft and readily redrawn using ordinary skill and this rich teaching. Accordingly, functional blocks, modules, subsystems, and the like can be combined, divided, repartitioned, redrawn, moved, reorganized, or otherwise altered without deviating from the scope and spirit of the disclosure. This is not to say that, nor will it support a conclusion that, the disclosed organizations and combinations are not novel, are not innovative, or are obvious.

Turning now to FIG. 1, this figure illustrates an example system 100 for dealing with breathing anomalies in a representative environment of a pediatric application, with the system represented in a functional block diagram format, according to some embodiments of the disclosure. As further discussed below, as illustrated in FIG. 1, the example system 100 provides a feedback loop for detecting and correcting breathing anomalies. Upon detection of a breathing anomaly or a determination that a breathing anomaly is about to occur, the system 100 can manipulate breathing to correct or avert the breathing anomaly. Operating in an example closed loop mode, the system 100 may issue a breathing correction upon detection of a start of a breathing anomaly and then issue additional corrections as needed, until the breathing anomaly is successfully suppressed, terminated, or suppressed.

As illustrated, the system 100 comprises a monitor 110 that monitors a subject 150 for an indication of an occurrence of a breathing anomaly, for example diminished breathing associated with sleep apnea. The monitor 110 comprises a sensor 105 that is positioned against the subject 150 via a wearable 115 and a receiver 111 that receives signals from the sensor 105. In some example embodiments, the sensor 105 senses one or more physiological parameters associated with breathing of the subject 150 (for example blood oxygen saturation, muscle activation, or cyclical movement of the subject's chest based on pressure or tension). The sensed one or more physiological parameters may provide an indication that the subject 150 is experiencing a breathing anomaly, for example near an onset of the anomaly. The sensed one or more physiological parameters may further be predictive of a breathing anomaly, for example comprising a precursor to an occurrence of a breathing anomaly. As illustrated, the sensor 105 and the receiver 111 are communicatively coupled to one another via a communication link 112. The communication link 112 between the sensor 105 and the receiver 111 may be wireless or wired. In some embodiments, the sensor 105 can be integrated into the monitor 110 to provide a unitary element in a common housing.

In the illustrated example of FIG. 1, the subject 150 is an infant. Other representative subjects may be toddlers, children, teenagers, adults, or elderly persons, for example. Embodiments of the illustrated system 100 can thus be beneficial for neonatal, pediatric, adult, and geriatric applications, for example.

The example wearable 115 of FIG. 1 comprises a strap that extends around the subject 150 and releasably fastens the sensor 105 to the subject 150. The wearable 115 illustrated in FIG. 1 is an example of an embodiment of a fastener. The term “fastener,” as used herein, refers to an apparatus or system that fastens something to something else, whether releasably, temporarily, or permanently. The wearable 115 orients the sensor 105 to sense pressure or motion associated with breathing. Thus, an example embodiment of the sensor 105 can comprise a pressure sensor. The sensor 105 transmits signals carrying physiological information to the receiver 111 via the communication link 112.

The receiver 111 receives and processes the signals and transmits resulting signals to the controller 125 via the sensor line 180, which can be wired or wireless. In some embodiments, the receiver 111 processes the signals to make a determination about whether the subject 150 is facing a breathing anomaly and then transmits the resulting determination to the controller 125. In other embodiments, the receiver 111 may merely process the sensor signals for reformatting or amplification or to convert from the wireless to wired domain, and then transmit the processed sensor signals to the controller 125 via the sensor line 180. In the latter embodiment, the controller 125 can make a determination about whether the subject 150 is facing a breathing anomaly.

As further discussed below, when a determination is made that the subject 150 is experiencing or otherwise facing a breathing anomaly, the controller 125 can initiate corrective action via the breathing stimulator 175. In the illustrated embodiment of FIG. 1, the controller 125 can send one or more signals to the breathing stimulator 175 to initiate corrective action.

Responsive to prompt by the controller 125, the breathing stimulator 175 stimulates a volar surface 160 of the subject 150. In the illustrated embodiment, the stimulated volar surface 160 is on a foot 170 of the subject 150. In other embodiments, the breathing stimulator can be positioned on a volar surface 160 of a hand 165 of the subject 150.

In some embodiments, the breathing stimulator 175 is positioned against a portion of a limb of the subject 150 other than the hand 165 or the foot 170. For example, the breathing stimulator 175 may be positioned to adjoin an anterior surface of a forearm of the subject 150 or a posterior surface of a lower leg of the subject 150 above the foot 170 of the subject 150. In some example embodiments, the selection of the placement of the breathing stimulator 175 is personalized, for example selected based on a sleep study that identifies an anatomical area where the subject 150 responds optimally or particularly well to breathing stimulation.

In some embodiments, the area of stimulation is varied so as to avoid acclimation or diminished responsiveness over time. For example, a particular subject 150 may utilize a regime whereby the breathing stimulator 175 is positioned on the right foot 170 during week one, the left foot 170 during week two, the right hand 165 during week three, the left hand 165 during week four, with the cycle repeating thereafter.

The term “volar surface,” as used herein, generally refers to a palmar surface of a hand, a surface of an arm located on same side of the arm as the palm, and/or a planar surface of a foot. The term “palmar surface of a hand,” as used herein, generally refers to the palm of the hand and includes the surfaces of the fingers located on the same side of the fingers as the palm. The term “planar surface of a foot,” as used herein, generally refers to the lower surface of the foot and includes the lower surfaces of the toes.

As further discussed below, in some embodiments, the system 100 can be applied to monitor for and document breathing anomalies during a sleep session. In some examples of such an application, the system 100 may operate without using the detection of a particular breathing anomaly as a trigger for intervening with a corrective action during the sleep session. The system 100 may be utilized in such a capacity during a sleep study, for example.

In some sleep study applications, the system 100 may further function in an open loop mode in which the system 100 stimulates the volar surface 160 cyclically while monitoring for breathing anomalies and subject responses. The system 100 may vary stimulation parameters over the course of several nights while recording the number of breathing anomalies that occur under the different parameters.

The parameters that prove most effective or otherwise best suited to the individual being studied can then be applied during routine sleep. For example, operating in open loop mode with these parameters, the system 100 can stimulate the volar surface 160 continuously, periodically, or according to a pre-defined program to provide a prophylactic against breathing anomalies.

Turning now to FIG. 2, this figure illustrates an example breathing monitor 110 that comprises a representative camera 200 according to some embodiments of the disclosure. The system 100 illustrated in FIG. 1 can incorporate, or be communicatively coupled to, the camera-based breathing monitor 110 illustrated in FIG. 2 as an alternative to monitoring breathing rate using a sensor 105 that contacts the subject 150 as discussed above. The term “communicatively coupled,” as used herein, refers to a state in which two or more components are connected such that communication signals are able to be exchanged between the components on a unidirectional or bidirectional (or multi-directional) manner, either wirelessly, through a wired connection, or a combination of both.

The camera 200 comprises a sensor 105 for capturing images of the subject 150 while the subject 150 is sleeping. The camera 200 can be sized for mounting to a bedframe, for example in a pediatric application. The captured images can be computer processed to determine breathing rate of the subject 150. The image processing can take place in the controller 125 or in the camera 200 itself, for example. The controller 125 can utilize the resulting breathing rate as a basis for implementing breathing corrections via stimulating the volar surface 160 of the subject 150, for example.

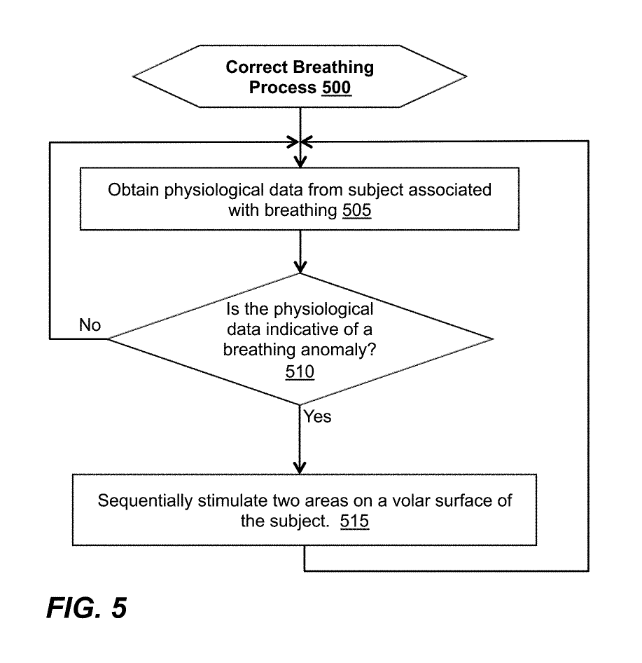

Turning now to FIGS. 3, 4, and 5, another example embodiment of a system 100 for dealing with breathing anomalies is illustrated and will be described in further detail. FIG. 3 illustrates an example wearable 115 that is configured to be worn on a foot 170 and that comprises a representative system 100 for dealing with breathing anomalies according to some embodiments of the disclosure. FIG. 4 illustrates a functional block diagram of an example system 100 for dealing with breathing anomalies according to some embodiments of the disclosure. The functional block diagram of FIG. 4 is applicable to the wearable 115 of FIG. 3 and will be discussed in such example context, without limitation. FIG. 5 illustrates a representative flowchart for an example process 500 for dealing with breathing anomalies according to some embodiments of the disclosure. The process 500 can be practiced with the system 100 illustrated in FIGS. 3 and 4 and will be discussed in such context, without limitation.

Referring now to FIG. 3, the illustrated wearable 115 is an example embodiment of a fastener that releasably fastens the system 100 to the subject 150. In accordance with the illustrated embodiment of FIG. 3, the system 100 can be attached to or integrated into a sock, sleeve, stocking, bootie, or close-fitting covering for a foot 170. Elements of the system 100 can be attached to a sock via sewing, adhesive, pouches, or other appropriate means, for example. In various embodiments, the system 100 can be attached to or integrated with various other articles of clothing, for example a shoe, a sandal, a glove, a mitt, or a wristband, to mention a few representative examples without limitation.

Referring now to FIGS. 3 and 4, the illustrated system 100 for dealing with breathing anomalies comprises a power supply 310, which can comprise a battery pack for example. The example monitor 110 comprises a toe-mounted pulse oximeter sensor 105 that senses blood oxygen saturation of the subject 150, for example based on light coupled into and returning back from the subject 150. Signals from the pulse oximeter sensor 105 transmit from an output of the monitor 110 (or the sensor 105) to the controller 125 over the sensor line 180. The controller 125 receives those signals via an input of the controller 125 and processes the signals to evaluate the subject 150 for breathing anomalies. When the results of the evaluation indicate need for breathing correction (for example when blood oxygen saturation crosses a threshold or moves outside a range), the controller 125 issues a control signal from an output of the controller 125. The control signal transmits to the breathing stimulator 175 over the control line 185. In response to receipt of the control signal, the breathing stimulator 175 stimulates or manipulates breathing.

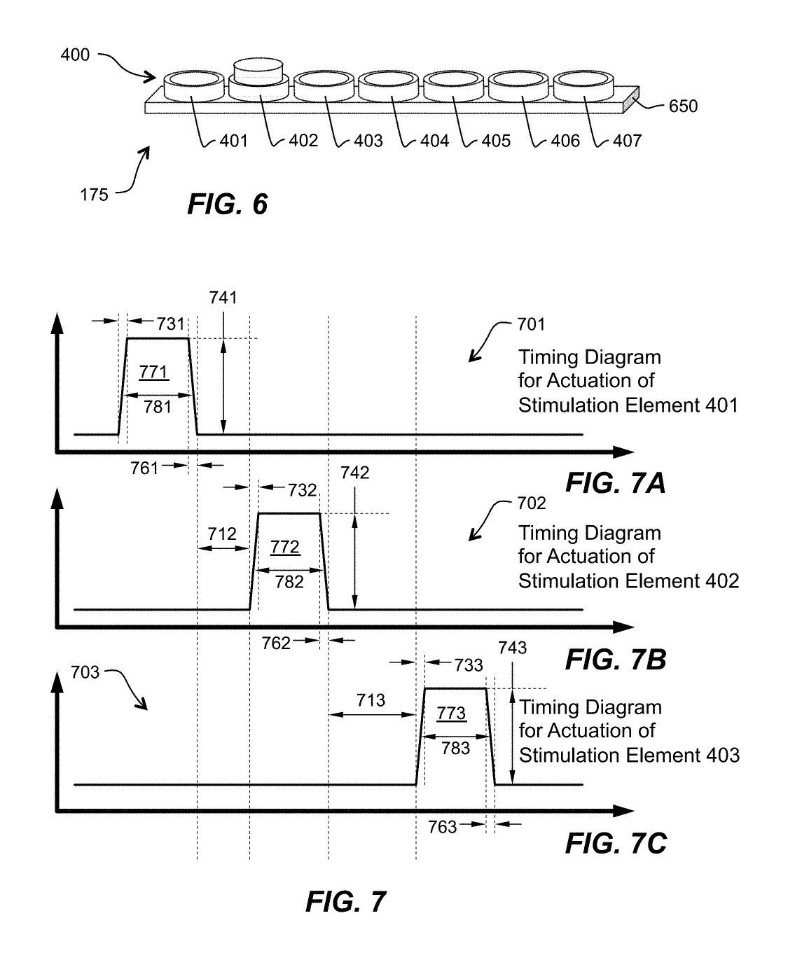

As further discussed below with reference to FIG. 6, the breathing stimulator can comprise an array of stimulation elements 400. The individual stimulation elements 401, 402, . . . of the array 400 can be arranged in a line or other appropriate geometric pattern and activated individually. In some example embodiments, the geometric pattern is two dimensional, so as to extend in two dimensions along the volar surface 160.

Referring now to FIG. 4, the controller 125 can comprise circuitry, logic, memory, and/or one or more digital controllers for storing and executing instructions. In the illustrated example embodiment, the controller 125 comprises or is operably coupled to a processor 405 and associated memory 410. As one of ordinary skill in the art will appreciate, the term “operably coupled,” as may be used herein, encompasses direct coupling and indirect coupling via another, intervening component, element, circuit, or module; moreover, a first component may be operably coupled to a second component when the first component comprises the second component.

As illustrated, the memory 410 stores a correct breathing engine 415 and associated settings 420 that may define stimulation settings personalized for a particular subject 150. The illustrated correct breathing engine 415 comprises computer-implemented instructions, which can persist in the memory 410, for execution by the processor 405. In an example embodiment, the breathing control engine 415 can comprise computer-implemented instructions for executing the process 500 for which FIG. 5 illustrates a representative flowchart as discussed below. The controller 125 can thus execute steps or blocks of process 500, which is denoted with the representative, non-limiting label of Correct Breathing Process.

In some example embodiments as illustrated in FIG. 4, in support of storing and executing instructions, the processor 405 can comprise a microprocessor, a microcontroller, a digital processor, or other appropriate circuitry or computer system. In certain example embodiments, the processor 405 can comprise logic implemented in hardware with any or a combination of the following technologies: a discrete logic circuit(s) having logic gates for implementing logic functions upon data signals, an application-specific integrated circuit (“ASIC”) having appropriate combinational logic gates, a programmable gate array(s) (“PGA”), a field programmable gate array (“FPGA”), etc.

In some example embodiments, the memory 410 illustrated in FIG. 4 can comprise assorted types of memory that may hold machine-executable instructions for dealing with breathing anomalies, and the instructions may persist in the memory. Forms of memory can include any one or combination of volatile memory elements (for example, forms of random access memory “RAM,” such as DRAM, SRAM, SDRAM, etc.), nonvolatile memory elements (for example, ROM, hard drive, tape, compact disc read-only memory (“CDROM”), etc.), and erasable memory (for example, erasable programmable read only memory (“EPROM”) and electrical EPROM (“EEPROM”)). The memory 410 can comprise persistent memory. The memory 410 can comprise non-transitory memory.

In certain embodiments, the controller 125 may incorporate electronic, magnetic, optical, and/or other types of storage media and can have a distributed architecture, where various components are situated remote from one another, but can be accessed over a network. Instructions and/or code for operating the controller 125, including routines associated with the process 500, the correct breathing engine 415, and the settings 420, can be stored in a persistent computer-readable medium.

A “computer-readable medium” can be any means that can store, communicate, propagate, or transport a program for use by or in connection with an instruction execution system, apparatus, or device. The computer-readable medium can comprise a persistent computer-readable medium. The computer-readable medium can comprise a non-transitory computer-readable medium.

The computer readable medium can be, for example but not limited to, an electronic, magnetic, optical, electromagnetic, infrared, or semiconductor system, apparatus, device, or propagation medium. More specific examples (a non-exhaustive list) of the computer-readable medium can include the following: an electrical connection (electronic) having one or more wires, a portable computer diskette (magnetic), a RAM (electronic), a read-only memory (“ROM”) (electronic), an erasable programmable read-only memory (EPROM, EEPROM, or Flash memory) (electronic), a data stick, a flash drive, and a portable CDROM (optical). Note that a computer-readable medium might even comprise a paper or another suitable medium upon which a program is printed, as the program may be electronically captured, via for instance optically scanning the paper or other medium, then compiled, interpreted or otherwise processed in a suitable manner if necessary, and then stored in a computer memory.

It should be apparent that one of ordinary skill in the art would be able to make and operate the controller 125 and the process 500 (as well as the other elements and embodiments disclosed herein) without difficulty and without undue experimentation based on the Figures, example functional block diagrams, flowcharts, and associated specification text. Therefore, additional disclosure of a particular set of program code instructions or more particularized circuit schematics is not considered necessary for an adequate understanding of how to make and use the present technology.

Referring now to the representative flowchart of FIG. 5, an example process 500 for dealing with breathing anomalies will be further described. Process 500 will be discussed with example reference to the embodiment of FIGS. 3 and 4. Process 500 can further be practiced with the embodiment illustrated in FIG. 1 as well as other embodiments disclosed herein. It will be appreciated that process 500 can operate with many embodiments and applications, without limitation.

Certain steps in process 500, as well as in the other processes and methods disclosed or taught herein, may naturally need to precede others to achieve desirable functionality. However, the disclosure is not limited to the order of the steps described if such order or sequence does not adversely alter functionality to the extent of rendering the technology inoperable or nonsensical. That is, it is recognized that some steps may be performed before or after other steps or in parallel with other steps without departing from the scope and spirit of the disclosure.

At block 505 of process 500, the monitor 110 obtains physiological data as discussed above. For example, the monitor 110 can sense blood oxygen saturation or breathing rate.

At inquiry block 510 of process 500, the system 100 determines whether the physiological data is indicative of a breathing anomaly. The determination can be carried out by the controller 125, by the monitor 110, or other appropriate devices, logic, or appropriate determination-making means.

Determining whether the physiological data is indicative of the breathing anomaly can comprise determining whether the physiological data meets one or more criteria that is indicative of the breathing anomaly, for example by comparing the physiological data to a threshold. In some example embodiments, determining whether the physiological data is indicative of the breathing anomaly can comprise monitoring breathing rate of the subject 150 and comparing the monitored breathing rate to a breathing-rate threshold. In some example embodiments, determining whether the physiological data is indicative of the breathing anomaly can comprise monitoring blood oxygen saturation of the subject 150 and comparing the monitored blood oxygen saturation to a blood-oxygen-saturation threshold.

If the determination of inquiry block 510 is negative, process 500 loops to block 505, executes block 505, and iterates until the determination is positive.

If the determination of inquiry block 510 is positive, process 500 executes block 515. At block 515, the controller 125 causes the breathing stimulator 175 to sequentially stimulate two areas on a volar surface 160 of the subject 150.

From block 515, process 500 loops to block 505 and iterates. In some example embodiments, process 500 provides a delay following the stimulation of block 515 in order to provide time for the stimulation to affect breathing of the subject 150.

Process 500 can monitor for breathing anomalies and issue breathing corrections in real-time, using a feedback loop. Process 515 can thus routinely deal with breathing anomalies of the subject 150, for example on a nightly basis while the subject 150 is sleeping.

In some embodiments, process 500 includes an alarm function. For example, if a breathing anomaly occurs or progresses despite stimulation by the breathing stimulator 175, the system 100 can raise an alarm. The system 100 may send notification to a caregiver's cell phone, for example, or otherwise issue an alarm. The caregiver can respond to the notification by waking the subject 150 to ensure sufficient breathing, for example. Process 500 can thus include a function for prompting human intervention when therapy is deemed insufficient or has not corrected breathing after a pre-determined time delay. In some example embodiments, the system 100 may raise an alarm when a predefined number of breathing corrections have been unsuccessful or inadequate.

In some example embodiments, a sleep study is conducted on the subject 150 separately from routine execution of process 500, with one or more intervening nights or days. For example, one or more sleep study sessions can be conducted on the subject 150 over one or more days or nights; and after study completion, the subject 150 can utilize the system 100 nightly to correct or otherwise manage breathing issues. In the sleep study, the subject 150 may be subjected to a range of stimulation settings in order to determine personalized stimulation settings or parameters that are most effective or otherwise best suited to the subject 150. The resulting settings may be loaded on the controller 125 and stored in the memory 410 as the settings 420, so that the system 100 provides personalized breathing stimulation.

In some embodiments, the settings 420 may be utilized in nightly practice of process 500 by the system 100, in feedback mode. In some other embodiments, the settings 420 may be utilized without the monitoring and real-time feedback provided by the flowchart of process 500. The settings 420 may be utilized to cyclically or repetitively stimulate the volar surface 160 of the subject 150 while the subject is sleeping, without utilizing detection of a breathing anomaly as a trigger. For example, the system 100 can be set to stimulate the volar surface 160 of the subject 150 periodically so as to preempt breathing anomalies. The period of time between the stimulations can be one of the settings 420 determined in the sleep study, for example. The system 100 can thus operate on a prophylactic basis that helps prevent the subject 150 from experiencing breathing anomalies.

In some embodiments of such prophylactic operation, the controller 125 issues a series of signals to the breathing stimulator 175 during the subject's routine sleep, with the series extending substantially over a sleep session. Responsive to receipt of each signal in the series, the breathing stimulator 175 can perform a stimulation, thereby providing repetitive breathing stimulation during the sleep session. Each performed stimulation can comprise sequential stimulation of two areas of the volar surface 160, for example.

In various embodiments, the breathing stimulator 175 can stimulate the volar surface 160 using force or movement directed into or substantially normal to the volar surface 160, force or movement directed substantially along or transverse to the volar surface 160, friction, rubbing, pressure, vibration, sound, heat, electricity, an electrical field, a magnetic field, electromagnetic waves, light, or other appropriate energy form (or combination thereof), to mention some representative examples without limitation. As one of ordinary skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, process variations, and manufacturing tolerance. Stimulating the volar surface 160 of the subject 150 to correct a breathing anomaly can comprise transmitting energy through an exterior layer of dead skin (in which the energy may not be directly sensed) and into underlying living tissue where the energy is sensed or otherwise produces a change in breathing of the subject 150.

In some example embodiments, the breathing stimulator 175 comprises a cylinder that has an elevated spiral surface to form a helix. The cylinder can be positioned adjacent the volar surface 160 with the outermost portion of the helix adjoining the volar surface 160. The cylinder rotates on command, via an electrical motor or other drive, to stimulate the volar surface 160. When the cylinder rotates, the point of contact between the helix and the volar surface 160 translates along the volar surface 160. The point of contact can thus sweep across the volar surface 160 to emulate a finger stroke and trigger the Babinski response. The figure labeled “Embodiment 2—Mechanical Spiral” and the accompanying text in U.S. Provisional Patent Application No. 62/432,609 (the entire contents of which are incorporated herein as provided above) describes and illustrates an example of such an embodiment.

In some example embodiments, the breathing stimulator 175 comprises a worm drive that translates an elevated hub across the volar surface 160. The translation can emulate a finger stroke and trigger the Babinski response. The figure labeled “Embodiment 3—Worm Gear Positioner” and the accompanying text in U.S. Provisional Patent Application No. 62/432,609 (the entire contents of which are incorporated herein as provided above) describes and illustrates an example of such an embodiment.

In some example embodiments, the breathing stimulator 175 comprises a shaft and an array of cams disposed along the length of the shaft, with associated cam followers. This system can be disposed adjacent the volar surface 160. When the shaft is rotated, the cams move the cam followers up and down. The cam followers sequentially press against the volar surface 160 to emulate a finger stroke and trigger the Babinski response. The figure labeled “Embodiment 4—Cam Gear Positioner” and the accompanying text in U.S. Provisional Patent Application No. 62/432,609 (the entire contents of which are incorporated herein as provided above) describes and illustrates an example of such an embodiment.

In some example embodiments, the breathing stimulator 175 comprises a belt drive that moves a member across the volar surface 160 and emulates a finger stroke to trigger the Babinski response. The figure labeled “Embodiment 5—Belt Drive” and the accompanying text in U.S. Provisional Patent Application No. 62/432,609 (the entire contents of which are incorporated herein as provided above) describes and illustrates an example of such an embodiment.

In some example embodiments, the breathing stimulator 175 comprises an array of stimulation elements 400 that are individually activated to affect breathing of the subject 150. Such embodiments will be further described below with reference to FIGS. 6 and 7.

Turning now to FIG. 6, this figure illustrates an example breathing stimulator 175 that comprises a representative array of stimulation elements 400 according to some embodiments of the disclosure. The example array of stimulation elements 400 illustrated by FIG. 6 has seven individual stimulation elements 401, 402, 403, 404, 405, 406, 407 arranged in a line, seven being an example number. Other embodiments may have fewer or more individual stimulation elements 401, 402, 403, 404, 405, 406, 407, for example two, that may be arranged in other geometric patterns.

In the illustrated example of FIG. 6, the individual stimulation elements 401, 402, 403, 404, 405, 406, 407 are mounted on a substrate 650, which can be flexible or rigid in various embodiments. The substrate 650 further may be flat or may be curved in some embodiments so as to substantially follow or conform to an arch of a sole of a foot 170, for example. The substrate 650 can be contoured according to contours of a volar surface 160 of a particular subject 150. In an example embodiment, the substrate 650 comprises a circuit board for distributing electricity to the individual stimulation elements 401, 402, 403, 404, 405, 406, 407.

In various embodiments, the individual stimulation elements 401, 402, 403, 404, 405, 406, 407 can comprise one or more piezoelectric devices, motors, solenoids, vibrators, laser diodes, light emitting diodes, speakers, electromagnets, antenna, electric field generators, electrodes, heating elements, or other appropriate devices. Each individual stimulation element 401, 402, 403, 404, 405, 406, 407 can receive electricity from the controller 125 and/or the power supply 310 and use the electricity to produce and delivery stimulation to a respective area of the volar surface 160. The controller 125 can thus activate or actuate the individual stimulation elements 401, 402, 403, 404, 405, 406, 407 on an element-by-element basis to deliver stimulation (which may be physical, force, movement, pressure, friction, electrical, magnetic, optical, thermal, electromagnetic, etc. as discussed above). When an individual stimulation element 401, 402, 403, 404, 405, 406, 407 is activated, the individual stimulation element 401, 402, 403, 404, 405, 406, 407 may move (for example into or towards the volar surface 160, across the volar surface 160, rotate, or oscillate) or may stimulate without movement (for example by emitting light or electricity into the volar surface 160). Thus, the stimulation elements 401, 402, 403, 404, 405, 406, 407 can be discrete devices, can apply discrete forces, and can be disposed at discrete locations on the volar surface 160, with the discrete locations collectively extending along the volar surface 160. The resulting stimulation can produce a stroking sensation in some example embodiments.

When the example breathing stimulator 175 that FIG. 6 illustrates is fastened to the subject 150 adjacent a volar surface 160, each of the individual stimulation elements 401, 402, 403, 404, 405, 406, 407 is oriented to stimulate a different area of the volar surface 160. In the illustrated example, each of the individual stimulation elements 401, 402, 403, 404, 405, 406, 407 comprises a discrete actuator, specifically a discrete solenoid. FIG. 6 thus illustrates an example embodiment of an array of actuators, in the form of an example array of solenoids. As illustrated, the stimulation element 402 is in an activated or actuated state for pressing upon or into an adjoining area of the volar surface 160.

Turning now to FIGS. 7A, 7B, and 7C, these figures illustrate example timing diagrams for individual stimulation elements 401, 402, 403 of a representative breathing stimulator 175, where the breathing stimulator 175 comprises an array of stimulation elements 400, according to some embodiments of the disclosure. FIG. 7 can be viewed as describing operation of a three-element array or operation of three representative elements in an array having more that three elements, such as illustrated in FIG. 6. More specifically, FIG. 7A illustrates activation or actuation of the stimulation element 401, FIG. 7B illustrates activation or actuation of the stimulation element 402, and FIG. 7C illustrates activation or actuation of the stimulation element 403. In the diagrams of FIGS. 7A, 7B, and 7C, the horizontal axis represents time.

As illustrated in FIGS. 7A, 7B, and 7C, a first pulse 771 activates or actuates breathing stimulation element 401, a second pulse 772 activates or actuates breathing stimulation element 402, and a third pulse 773 activates or actuates breathing stimulation element 403. There is a time delay 712 between pulse 771 and pulse 772 and another time delay 713 between pulse 772 and pulse 773. The two time delays 712, 713 may be equal in some embodiments, with the delay value selected for personalization as discussed above. In other embodiments, the two time delays 712, 713 may be different and selected for personalization, such as to provide an optimized breathing response based on a sleep study as discussed above. Thus, the resulting movements can provide sequential pressures or touches that have an off-periodicity and an on-periodicity, with the periodicities equal in some embodiments and different in others.

In some embodiments, there is substantially no time delay between the three pulses 771, 772, 773 to provide a wave of stimulation moving substantially fluidly across the volar surface 160 of the subject 150. For example, the pulses 771, 772, 773 may partially overlap in time, so that as one solenoid rod retracts, an adjacent solenoid rod extends. The result of such coordinated retraction and extension can promote a stoking sensation in some example embodiments.

Each of the pulses 771, 772, 773 has a respective pulse amplitude 741, 742, 743. The pulse amplitudes 741, 742, 743 may be equal in some embodiments. In other embodiments, the individual pulse amplitudes 741, 742, 743 may be different from one another and selected for personalization, such as to provide an optimized breathing response based on a sleep study as discussed above. The pulse amplitudes 741, 742, 743 may correspond to degree of extension of a solenoid rod or to applied pressure or to amount of stimulation applied, for example.

Each of the pulses 771, 772, 773 has a respective pulse width 781, 782, 783. The pulse widths 781, 782, 783 may be equal in some embodiments. In other embodiments, the individual pulse widths 781, 782, 783 may be different from one another and selected for personalization, such as to provide an optimized breathing response based on a sleep study as discussed above.

Each of the pulses 771, 772, 773 has a respective rise time 731, 732, 733 and a respective fall time 761, 762, 763. The respective rise times 731, 732, 733 can specify rate of activation or actuation, for example how fast a solenoid rod extends and presses against the volar surface 160. The respective fall times 761, 762, 763 can specify rate of deactivation or de-actuation, for example how fast a solenoid rod retracts and releases pressure from the volar surface 160.

The respective rise times 731, 732, 733 and fall times 761, 762, 763 may be equal in some embodiments. In other embodiments, the individual rise times 731, 732, 733 and fall times 761, 762, 763 may be different from one another and selected for personalization, such as to provide an optimized breathing response based on a sleep study as discussed above.

The time delays 712, 713; the pulse amplitudes 741, 742, 743; the pulse widths 781, 782, 783; the activation rates or rise times 731, 732, 733; and the deactivation rates or fall times 761, 762, 763 are example embodiments of the settings 420 for the controller 125 that can be stored in memory 410 as discussed above with reference to FIG. 4. The settings 420 can thus comprise stimulation parameters selected on a subject-by-subject basis.

The breathing stimulator 175 and the controller 125 can be operable across a range of stimulation settings. The memory 410 can be configured to store one or more settings 420 from the range that are selected based on personal sensitivity of the subject 150 to breathing stimulation in connection with correcting breathing anomalies. As discussed above, the breathing stimulator 175 can comprise an array of actuators in some example embodiments. In such an embodiment, the selected stimulation settings can comprise a stimulation setting selected based on personal sensitivity of the subject 150 to breathing stimulation by actuation of an actuator when the subject 150 is asleep and wearing the wearable 115.

Technology useful for dealing with breathing anomalies has been described. From the description, it will be appreciated that an embodiment of the disclosure overcomes limitations of the prior art. Those skilled in the art will appreciate that the technology is not limited to any specifically discussed application or implementation and that the embodiments described herein are illustrative and not restrictive. Furthermore, the particular features, structures, or characteristics that are set forth may be combined in any suitable manner in one or more embodiments based on this disclosure and ordinary skill. Those of ordinary skill having benefit of this disclosure can make, use, and practice a wide range of embodiments via combining the disclosed features and elements in many permutations without undue experimentation. This disclosure not only includes the illustrated and described embodiments, but also provides a rich and detailed roadmap for creating many additional embodiments using the various disclosed technologies, elements, features, and their equivalents. From the description of the example embodiments, equivalents of the elements shown herein will suggest themselves to those skilled in the art, and ways of constructing other embodiments will appear to practitioners of the art. Therefore, the scope of the technology is to be limited only by the appended claims.

What is claimed is:

1. A method for breathing manipulation, comprising the steps of:
determining whether breathing of a subject meets one or more criteria;
if the breathing of the subject meets the one or more criteria, then issuing a signal to an apparatus disposed adjacent a volar surface of the subject; and
responsive to the apparatus receiving the issued signal, sequentially stimulating, by the apparatus, a first area and a second area on the volar surface of the subject to manipulate the breathing of the subject.

2. The method of claim 1, wherein the step of determining whether the breathing of the subject meets the one or more criteria comprises
conducting a study on the subject in at least one first session,
wherein the step of if the breathing of the subject meets the one or more criteria, then issuing the signal to the apparatus disposed adjacent the volar surface of the subject comprises
issuing a series of signals to the apparatus during a second session while the subject is sleeping, the second session separated from the at least one first session by at least one day, and
wherein the step of responsive to the apparatus receiving the issued signal, sequentially stimulating, by the apparatus, the first area and the second area on the volar surface of the subject to manipulate the breathing of the subject comprises
for each signal in the series, sequentially stimulating the first area and the second area on the volar surface of the subject, thereby providing repetitive stimulation during the second session.

3. The method of claim 1, wherein the method further comprises, during a sleep session, iterating the steps of:
determining whether the breathing of the subject meets the one or more criteria;
if the breathing of the subject meets the one or more criteria, then issuing the signal to the apparatus disposed adjacent the volar surface of the subject; and
responsive to the apparatus receiving the issued signal, sequentially stimulating, by the apparatus, the first area and the second area on the volar surface of the subject to manipulate the breathing of the subject,
wherein determining whether the breathing of the subject meets the one or more criteria comprises:
monitoring breathing rate of the subject and comparing the monitored breathing rate to a breathing rate threshold; or
monitoring blood oxygen saturation of the subject and comparing the monitored blood oxygen saturation to a blood oxygen saturation threshold.

4. The method of claim 1, wherein sequentially stimulating, by the apparatus, the first area and the second area comprises the apparatus producing a stroking sensation extending across the first area and the second area on the volar surface of the subject.

5. The method of claim 1, wherein sequentially stimulating, by the apparatus, the first area and the second area comprises the apparatus sequentially applying discrete forces upon the volar surface at discrete locations that collectively extend along the volar surface.

6. The method of claim 1, wherein the apparatus comprises an array of actuators disposed adjacent the volar surface of the subject, and
wherein the step of responsive to the apparatus receiving the issued signal, sequentially stimulating, by the apparatus, the first area and the second area on the volar surface of the subject to manipulate the breathing of the subject comprises
sequentially actuating each actuator in the array.

7. The method of claim 6, wherein the array of actuators comprises a first actuator, a second actuator, and a third actuator, with the second actuator disposed between the first actuator and the third actuator, and
wherein sequentially activating each actuator in the array comprises:
activating the first actuator;
upon passage of a first amount of time following activation of the first actuator, deactivating the first actuator;
upon passage of a second amount of time following deactivation of the first actuator, activating the second actuator;
upon passage of a third amount of time following activation of the second actuator, deactivating the second actuator;
upon passage of a fourth amount of time following deactivation of the second actuator, activating the third actuator; and
upon passage of a fifth amount of time following activation of the third actuator, deactivating the third actuator.

8. The method of claim 1, wherein sequentially stimulating the first area and the second area on the volar surface of the subject comprises producing the Babinski response.

9. A system for breathing stimulation, comprising:
a monitor comprising:
a sensor configured to sense one or more physiological parameters associated with breathing of a subject; and
a first output configured to output at least one first signal based on the sensed one or more physiological parameters;
a controller that is operably coupled to the monitor and that comprises:
an input configured to receive the at least one first signal;
a processor configured to process the at least one first signal and produce at least one second signal when the at least one first signal indicates diminished breathing of the subject; and
a second output configured to output the at least one second signal; and
a stimulator that is operably coupled to the controller and that comprises:
an array of actuators that actuate sequentially responsive to receipt of the at least one second signal via the second output; and
a fastener that is sized for releasably disposing the array of actuators adjacent a volar surface of the subject so that the actuators of the array are at different locations along the volar surface.

10. The system of claim 9, wherein the array of actuators comprises an array of solenoids or piezoelectric devices, and the solenoids or piezoelectric devices are configured to emulate a finger stroke across the volar surface when sequentially activated.

11. The system of claim 9, wherein sensing one or more physiological parameters associated with breathing of the subject comprises sensing blood oxygen saturation or breathing rate.

12. The system of claim 9, wherein the fastener comprises at least one of a shoe, a bootie, a sock, a stocking, a sandal, a glove, a mitt, a strap, an article of clothing, and a band.

13. The system of claim 9, wherein the monitor comprises a camera sized for mounting adjacent a bed of the subject,
wherein the system further comprises a battery connected to supply power to at least the stimulator, and
wherein the stimulator comprises a wearable.

14. The system of claim 9, wherein the array of actuators comprises a plurality of actuators disposed in a geometric pattern,
wherein the controller is further configured to actuate the plurality of actuators in a sequence, with each actuator actuated for a respective time period, and
wherein the geometric pattern, the sequence, and the respective time periods are each selected for producing the Babinski response.

15. The system of claim 9, wherein the array of actuators comprises a first actuator, a second actuator, and a third actuator, with the second actuator disposed between the first actuator and the third actuator, and
wherein actuating sequentially responsive to receipt of the at least one second signal via the second output comprises:
actuating the first actuator at a first rate;
upon passage of a first amount of time following actuation of the first actuator, retracting the first actuator at a second rate;
upon passage of a second amount of time following retraction of the first actuator, actuating the second actuator at a third rate;
upon passage of a third amount of time following actuation of the second actuator, retracting the second actuator at a fourth rate;
upon passage of a fourth amount of time following retraction of the second actuator, actuating the third actuator at a fifth rate; and
upon passage of a fifth amount of time following actuation of the third actuator, retracting the third actuator at a sixth rate.

16. The system of claim 15, wherein the controller is configured to store the first amount of time, the second amount of time, the third amount of time, the fourth amount of time, the fifth amount of time, the first rate, the second rate, the third rate, the fourth rate, the fifth rate, and the sixth rate with values selected for breathing stimulation.

17. The system of claim 15, wherein the controller comprises a memory, and
wherein the memory is configured to store stimulation parameters for the array of actuators that are selected on a subject-by-subject basis for correcting breathing anomalies associated with sleep apnea.

18. A wearable comprising:
a power supply;
an actuator that is operably coupled to the power supply;
a fastener configured for releasably fastening the actuator to a limb of a subject so that the actuator is disposed adjacent a surface of the limb;
a controller that is operably coupled to the power supply and to the actuator, the controller operable to control the actuator across a range of stimulation settings; and
a memory that is configured for storing a selected stimulation setting from the range of stimulation settings, the memory operably coupled to the controller so that the controller actuates the actuator according to the stored, selected stimulation setting,
wherein the selected stimulation setting is selected based on personal sensitivity of the subject to breathing stimulation via actuation of the actuator with the fastener releasably fastening the actuator to the limb of the subject with the subject asleep.

19. The wearable of claim 18, wherein the selected stimulation setting is selected based on a sleep study conducted on the subject.

20. The wearable of claim 18, wherein the actuator comprises an array of solenoids that is configured to produce a stroking sensation on the subject, and
wherein the controller is further operable to:
provide an open loop mode and a closed loop mode, and
issue an alarm based on a determination that an occurrence of one or more breathing anomalies warrants caregiver intervention.
